(19) United States
(12) Patent Application Publication
(10) Pub. No.: US 2010/0057013 A1
(43) Pub. Date: Mar. 4, 2010

(54) INTRACORPOREAL INDWELLING EQUIPMENT

(75) Inventor: Hisataka Harada, Fukuroi-shi (JP)

Correspondence Address: TYCO HEALTHCARE - EDWARD S. JARMOLOWICZ, 15 HAMPSHIRE STREET, MANSFIELD, MA, 02048, US

(73) Assignee: TYCO HEALTHCARE GROUP LP, Mansfield, MA (US)

(21) Appl. No.: 11/911,936

(22) PCT Filed: Apr. 21, 2006
(86) PCT No.: PCT/EP06/03716

§ 371 Date: Aug. 14, 2009

(30) Foreign Application Priority Data
Apr. 21, 2005 (JP) 2005-123793

(51) Int. Cl. A61J 15/00; A61M 25/04

(52) U.S. Cl. 604/175

(57) ABSTRACT

The present invention provides an intracorporeal indwelling equipment having an internal holding member to be set in the stomach. Said internal holding member is composed of flexible ribbon-shaped members and flexible membrane-like members that are set on the portions of ribbon-shaped member, forming a dome-shaped portion. In the center of the outer periphery of membrane-like member, a notch and folding creases are formed to assist in deployment and help prevent the generation of ulcers and buried bumper syndrome.

### FIELD OF THE INVENTION

The present invention pertains to a type of intracorporeal indwelling equipment for feeding fluid, such as liquid food or the like, into the stomach or another prescribed portion of a patient, or for exhausting urine or other fluid waste from renal pelvis or another prescribed portion of the body of a patient.

### BACKGROUND OF THE INVENTION

In the prior art, for a person who cannot take food by mouth on his/her own due to age or disease (hereinafter to be referred to as the patient), intracorporeal indwelling equipment is used to feed liquid food, nutrients or other fluids. The intracorporeal indwelling equipment is composed of the following parts: a cylindrical member inserted into the fistula set in the body of the patient for taking the liquid food, an internal holding member attached on the tip of the cylindrical member and inserted into the stomach wall of the patient, and an external holding member attached on the outer peripheral surface of the cylindrical member and set on the skin surface side of the body of the patient (for example, see Japanese Kokai Patent Application No. Hei 4[1992]-303461). For the intracorporeal indwelling equipment, the internal holding member is made of a triangular flange-like member set on the outer peripheral surface of a cylindrical member. See also Applicant's earlier W02005/105018 (not published at the priority date declared herein).

For said conventional intracorporeal indwelling equipment, because of degradation that takes place for a prescribed period as well as change in the thickness of the stomach wall corresponding to increase in the body weight and growth of the patient, the indwelling equipment should be replaced after each prescribed period. However, for the aforementioned conventional intracorporeal indwelling equipment, because the internal holding member spreads in the direction perpendicular to the cylindrical member, it is difficult to perform the operation of insertion into/withdrawing from the fistula of the patient. Also, because the internal holding member is formed in a flat, flange-like shape, the corners of the internal holding member stimulate the stomach wall, which may generate ulcers. This is undesirable. It may also generate buried bumper syndrome. Here, the buried bumper syndrome refers to the state in which the holding force between the internal holding member and the external holding member becomes higher, the tissues of the stomach wall and abdominal wall become weaker, and the internal holding member is gradually buried in the stomach wall and abdominal wall.

An objective of the present invention is to solve the aforementioned problems of the prior art by providing a type of intracorporeal indwelling equipment characterized by the fact that it facilitates insertion into/withdrawing from the fistula of the patient, and that it can prevent generation of ulcers or buried bumper syndrome.

### SUMMARY OF THE INVENTION

In an embodiment, the present invention provides a type of intracorporeal indwelling equipment comprising a cylindrical member set in the fistula formed between the skin side wall portion on the body of the patient and the wall portion of the prescribed portion in the body of the patient, and an internal holding member that is connected to the tip of said cylindrical member and is set on the inner side of said prescribed portion, to permit fluid flow between the exterior of said patient and the interior of said prescribed portion; said internal holding member is composed of multiple flexible ribbon-shaped members, each bending from the opening edge at the tip of said cylindrical member towards the outer side and bonded to each other at their tips, and flexible membrane-like members set on the portions of said multiple ribbon-shaped members on said cylindrical member side, and forming a dome-like portion together with said multiple ribbon-shaped members; a notch is formed on the outer periphery of each membrane-like member of said dome-like portion; when said multiple ribbon-shaped members are stretched so that the tip-bonding portions of said ribbon-shaped members separate from said cylindrical member, said membrane-like members are folded with said notch taken as the base of the crease.

### BRIEF DESCRIPTION OF DRAWINGS

For a better understanding of the present invention, and to show more clearly how the same may be carried into effect, reference will now be made, by way of example, to the accompanying drawings, in which:

FIG. 1 is a cross-sectional view illustrating the intracorporeal indwelling equipment in the first embodiment of the present invention.

FIG. 2 is a plan view of the internal holding member in the intracorporeal indwelling equipment shown in FIG. 1.

FIG. 3 is a front view of the internal holding member shown in FIG. 2.

FIG. 4 is a side view of the internal holding member shown in FIG. 2.

FIG. 5 is a front view illustrating the state in which the intracorporeal indwelling equipment is stretched by the stretching unit.

FIG. 6 is a front view illustrating the extender.

FIG. 7 is a comparative example of the extender.

FIG. 8 is a plan view of the stretching auxiliary unit.

FIG. 9 is a front view of the stretching auxiliary unit.

FIG. 10 is a side view of the stretching auxiliary unit.

FIG. 11 is a plan view illustrating the stretched state of the internal holding member.

FIG. 12 is a cross-sectional view illustrating the intracorporeal indwelling equipment in the second embodiment of the present invention.

FIG. 13 is a plan view of the internal holding member in the intracorporeal indwelling equipment shown in FIG. 12.

FIG. 14 is a front view of the internal holding member shown in FIG. 13.

FIG. 15 is a side view of the internal holding member shown in FIG. 13.

FIG. 16 is a front view illustrating the state in which the intracorporeal indwelling equipment is stretched by a stretching unit shown in FIG. 12.

FIG. 17 is a plan view of the internal holding member in the intracorporeal indwelling equipment in the third embodiment of the present invention.

FIG. 18 is a plan view illustrating the state in which the internal holding member shown in FIG. 17 is stretched.

### DETAILED DESCRIPTION OF THE INVENTION

### First Embodiment

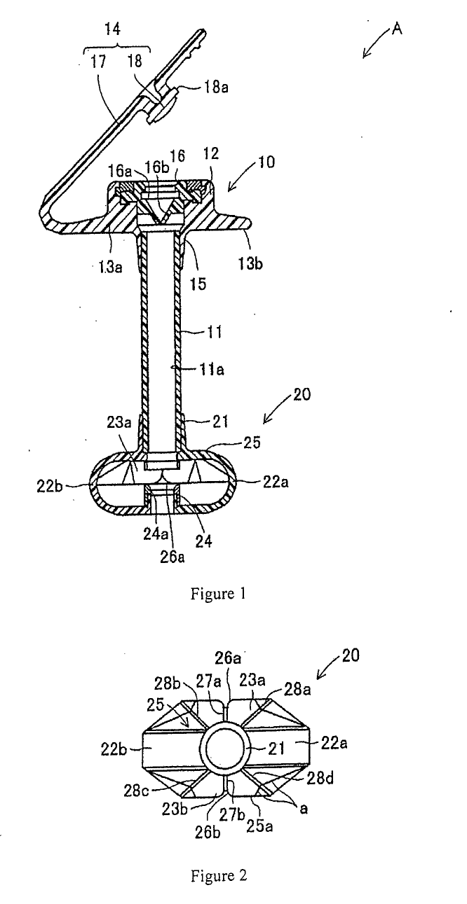

In the following, an explanation will be given regarding first embodiment of the present invention. FIG. 1 shows intracorporeal indwelling equipment A of this embodiment. This intracorporeal indwelling equipment A is set in the fistula formed between the abdomen and stomach wall of the patient for feeding fluids, such as liquid food or the like, into the stomach. It is composed of external holding member (10), cylindrical member (11) connected to the lower end of external holding member (10), and internal holding member (20) attached on the lower end of cylindrical member (11), all made of polyurethane. In the following, an explanation will be given regarding the case when external holding member (10) is on the upper side and internal holding member (20) is on the lower side.

Said external holding member (10) is composed of main body (12) formed in a rather thick ring shape, a pair of external holding pieces (13a), (13b) protruding outward from the lower end portions of the left/right sides of main body (12), lid member (14) integrally connected to one external holding piece (13a), and cylindrical connecting portion (15) formed at the center of the lower surface of main body (12). On the peripheral surface of coupling hole (16) formed through in the vertical direction at the center of main body (12), coupling crease (16a) is formed along the circumference.

Said coupling hole (16) has a shape that is wider on the upper opening side and tapers as the position of the lower portion of coupling crease (16a) goes downward. On the lower end portion of coupling hole (16), check valve (16b) is formed with a circular upper end opening and linear lower end inflow port. A space is formed on the outer peripheral side of said check valve (16b). Also, external holding pieces (13a), (13b) extend horizontally from the side of main body (12), and, together with main body (12), they work to prevent intracorporeal indwelling equipment A from being sucked into the stomach.

Said lid member (14) is composed of ribbon-shaped connecting portion (17) connected to external holding piece (13a) and stopper portion (18) set on the tip side portion of ribbon-shaped connecting portion (17). Said ribbon-shaped connecting portion (17) is flexible, and it can be bent to rotate vertically or bent at a large angle around the connecting portion with external holding piece (13a) at the center. Said stopper portion (18) is set at a position facing coupling hole (16) when ribbon-shaped connecting portion (17) is bent to have its tip side portion positioned above main body (12). Said stopper portion (18) is formed in a short cylindrical shape with a length that allows coupling to coupling hole (16), and, on its outer peripheral surface, protrusions (18a) are formed along the circumference that can engage with crease (16a) on coupling hole (16) in a quick-connect/disconnect manner.

Consequently, when ribbon-shaped connecting portion (17) is bent and stopper portion (18) is pressed in coupling hole (16), it is possible to engage crease (16a) and protrusions (18a). As a result, coupling hole (16) of main body (12) can be closed. Also, when the tip of ribbon-shaped connecting portion (17) is pulled to release the coupling between stopper portion (18) and coupling hole (16), coupling hole (16) of main body (12) can be opened. Said connecting portion (15) extends downward from the periphery of the space on the outer periphery of check valve (16b), and the upper end portion of cylindrical member (11) is inserted into it and fixed. At the center of said cylindrical member (11), feed flow path (11a) is formed so that the liquid food or other fluids (not shown in the figure) can pass through it, and the upper end of feed flow path (11a) is connected via check valve (16b) to coupling hole (16) of external holding member (10).

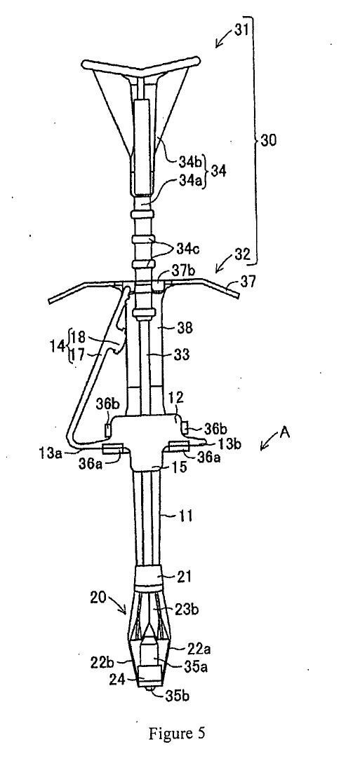

Said internal holding member (20) has the structure shown in FIGS. 2-4, and it is composed of cylindrical connecting portion (21), two ribbon-shaped members (22a), (22b), membrane-like members (23a), (23b), and coupling member (24). Said connecting portion (21) forms the upper end portion of internal holding member (20), and it is fixed while the lower end portion of cylindrical member (11) is inserted into it. Said ribbon-shaped members (22a), (22b) are connected to the two side portions on the lower end peripheral surface of connecting portion (21). After extending outward from the facing portions on the outer peripheral surface of connecting portion (21), they are bent to form a curve extending from the lower side to the lower side of cylindrical member (11).

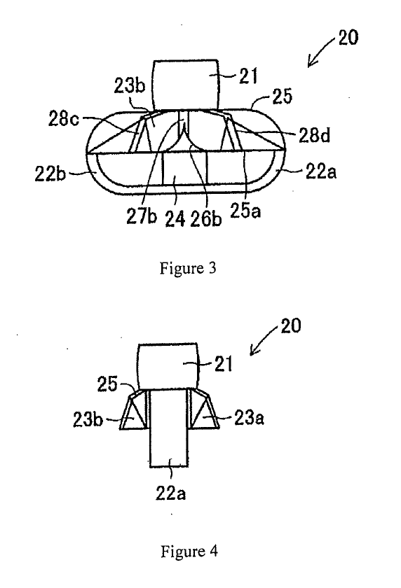

That is, the lower end portions of ribbon-shaped members (22a), (22b) are collected and fixed on the lower portion of the central axis of cylindrical member (11). As shown in FIGS. 1 and 3, ribbon-shaped members (22a), (22b) are formed facing each other to depict an ellipse with its minor axis in the longitudinal direction. Also, said ribbon-shaped members (22a), (22b) are flexible. Usually, by means of the elasticity, an overall elliptical shape is maintained. However, as the lower end portion is pulled downward, it stretches to a nearly linear shape. Then, when the withdrawing force is removed, it recovers the original elliptical shape.

Said membrane-like members (23a), (23b) are formed facing each other in the upper side portion in between ribbon-shaped members (22a), (22b), and they form together with the upper side portion of ribbon-shaped members (22a), (22b) the contact portion (25) in dome shape having a nearly elliptical plan view. The portion in contact with the stomach wall of said contact portion (25) formed in a dome shape is formed in a nearly planar shape. Also, said membrane-like members (23a), (23b) are flexible thin membranes, and they stretch under the external force.

Consequently, when ribbon-shaped members (22a), (22b) deform under external forces, membrane-like members (23a), (23b) follow the deformation of ribbon-shaped members (22a), (22b) and also deform. Also, notches (26a), (26b) are formed at the central portion of the outer periphery (25a) (lower end edge) of membrane-like members (23a), (23b). Said notches (26a), (26b) are formed in a nearly triangular shape with a larger width at the lower side and tapering narrower as the position goes upward. The edges on the two sides forming the triangular shape are formed in an arc shape and protrude from the central side portion so that notches (26a), (26b) become narrower.

Then, creases (27a), (27b) for folding are formed as short, thin, linear portions of the present invention extending from the upper end portions of notches (26a), (26b) to the lower end portion of connecting portion (21). Also, creases (28a), (28b), (28c), (28d) for folding are formed as the long, thin, linear portions of the present invention from the outer periphery (25a) of membrane-like members (23a), (23b) to the lower end of connecting portion (21) nearly to the central portion of notches (26a), (26b) on outer periphery (25a) of membrane-like members (23a), (23b) and their adjacent ribbon-shaped members (22a), (22b), and the lower end portion of the corresponding connecting portion (21). Said crease (28a), etc., for folding is formed along the dome shape of membrane-like member (23a), etc., so that they are bent in the upper and lower portion sides.

That is, for the shape of membrane-like members (23a), (23b), creases (27a), (27b) for folding are on the trough side, and, for creases (28a), (28b), (28c), (28d) for folding, the upper side portion is on the crest side, and the lower side portion is on the trough side. Also, the portions of lines (a) that connect the boundary portions between the crest side portion and trough side portion in said creases (28a), (28b), (28c), (28d) for folding and the portions of outer periphery (25a) of membrane-like members (23a), (23b) on the side of notches (26a), (26b) and on the side of ribbon-shaped members (22a), (22b) become the crest side in the structure formed.

Consequently, when a prescribed force is applied on membrane-like members (23a), (23b), membrane-like members (23a), (23b) are folded along the direction that connects notches (26a), (26b) and creases (27a), (27b) and along creases (28a), (28b), (28c), (28d). Said creases (27a), etc., have a structure with thin linear portions formed in the prescribed portions of membrane-like member (23a), etc.

Also, coupling member (24) is formed in a short cylindrical shape in the axial direction. By means of inserting plastic molding, it is fixed on the lower end portion of ribbon-shaped members (22a), (22b). That is, for coupling member (24), by connecting the lower end portions of ribbon-shaped members (22a), (22b) on the side lower end portion, ribbon-shaped members (22a), (22b) are connected to each other and, at the same time, its position is located below the central axis of cylindrical member (11) by ribbon-shaped members (22a), (22b). Also, in the central portion on the upper end of coupling member (24), hole (24a) is formed for positioning the tip of extender (31) (see FIG. 5). The inner periphery of said hole (24a) has a slope formed on it with the upper side having a larger diameter. It is positioned such that the tip of extender (31) is in contact with the slope surface.

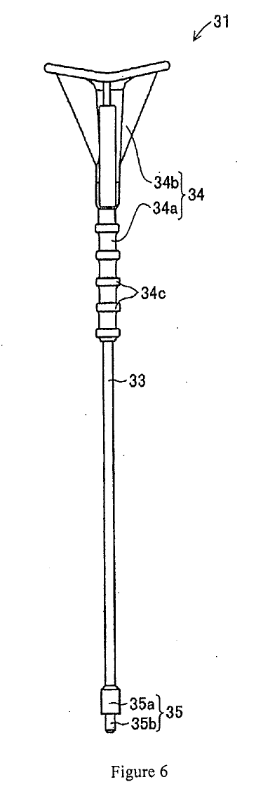

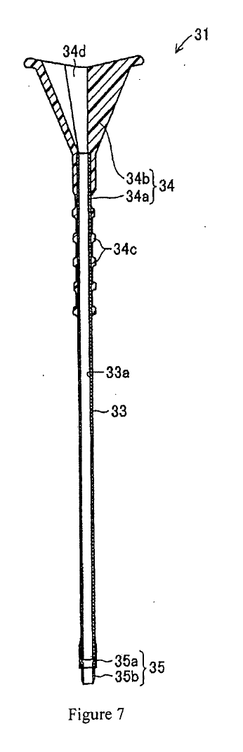

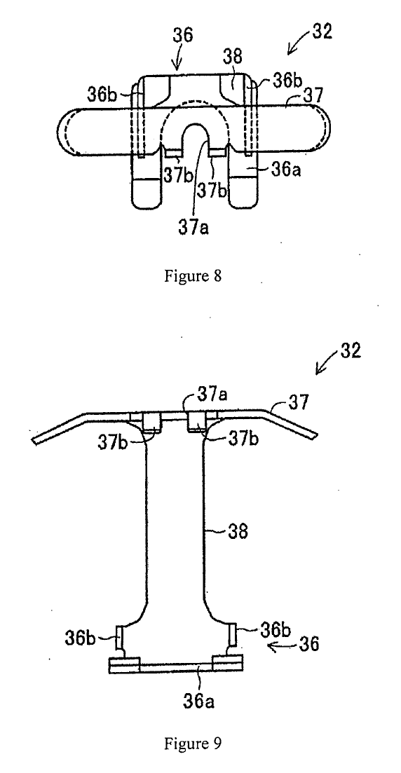

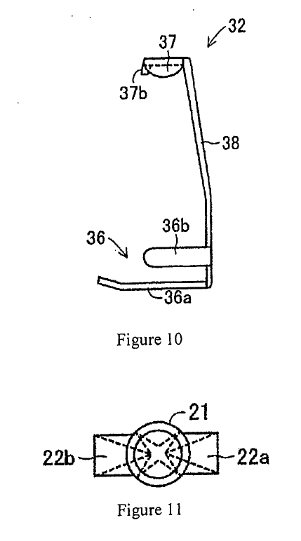

As shown in FIG. 5, when intracorporeal indwelling equipment A with said constitution is used, intracorporeal indwelling equipment A stretches by means of extending unit (30). Said extending unit (30) is composed of extender (31) shown in FIGS. 6 and 7 and cylindrical portion (32) shown in FIGS. 8-10. Said extender (31) has cylindrical main body (33) with internal cavity (33a) made of a stainless steel cylindrical body and formed for passing a guide wire (not shown in the figure) through it, and grip (34) made of plastic material. Then, on the lower end of cylindrical main body (33), a chip (35) made of plastic material is attached.

Said grip (34) is formed as a hand-hold extender (31), and it is composed of coupling portion (34a) fixed on cylindrical main body (33) while the upper outer periphery of cylindrical main body (33) is covered, and holding portion (34b) is integrally formed to coupling portion (34a). On the outer peripheral surface of coupling portion (34a), five step portions (34c) are formed with a prescribed vertical spacing. Said step portions (34c) are formed by ring-shaped protrusions with a semicircular cross-sectional shape set along the circumference of coupling portion (34a).

As shown in FIGS. 6 and 7, said holding portion (34b) is formed in a nearly triangular shape with the width in the left/right direction tapered larger as the position moves up. In its interior, inserting hole (34d) that connects to lumen (33a) of cylindrical main body (33) is formed in the vertical direction. Said inserting hole (34d) is formed in nearly triangular shape with the upper side portion of one side portion (the left side portion in FIG. 7) from the central portion in the left/right direction of holding portion (34b) becoming wider. On the upper surface of holding portion (34b), a curved surface with arc-shaped recess is formed to facilitate fitting of the hand and fingers during operation of extender (31).

Chip (35) is composed of fixing portion (35a) fixed on cylindrical main body (33) while the peripheral surface of the lower end portion of cylindrical main body (33) is covered, and press-in piece (35b) extends from the lower end of fixing portion (35a) downward. The outer diameter of fixing portion (35a) is selected to be larger than the diameter of hole (24a) of coupling member (24), and the diameter of press-in piece (35b) is selected to be smaller than the diameter of hole (24a). Consequently, when extender (31) is inserted downward from coupling hole (16) of intracorporeal indwelling equipment A, press-in piece (35b) enters the hole (24a) of coupling member (24), and, for fixing portion (35a), the lower surface becomes positioned on the upper surface of coupling member (24). Consequently, when extender (31) is pressed downward into intracorporeal indwelling equipment A, as shown in FIG. 5, internal holding member (20) becomes slender and extends.

Extending auxiliary member (32) is formed by processing a stainless steel sheet. It is composed of lower coupling portion (36), upper coupling portion (37) and connecting piece (38) in rectangular shape extending vertically and connecting lower coupling portion (36) with upper coupling portion (37). Said lower coupling portion (36) is composed of holding piece (36a) having a nearly U-shape in the plan view and formed perpendicular to connecting piece (38) and extending horizontally towards the front side as shown in FIG. 9, and a pair of hooks (36b) that are perpendicular to connecting piece (38) and holding piece (36a) and are set parallel to each other with a prescribed spacing from holding piece (36a). Also, the nearly U-shaped inner portion of holding piece (36a) is formed in the recess with a size appropriate for inserting of connecting portion (15) of external holding member (10), and the spacing between holding piece (36a) and hooks (36b) is selected to be size an appropriate for holding external holding pieces (13a), (13b).

Said upper coupling portion (37) is formed as a lateral plate member extending from the upper end portion of connecting piece (38), perpendicular to connecting piece (38) and towards the front-side horizontal direction shown in FIG. 9, and extending to the directions of both sides of connecting piece (38). Also, the length in the longitudinal direction of upper coupling portion (37) is selected to be short, and coupling recess (37a) that can be coupled to each step portion (34e) of extender (31) is formed in the central front portion. Also, on the two side portions of coupling recess (37a) in the front portion of upper coupling portion (37), a pair of downward protrusions (37b) is formed for preventing the release of the coupling with step portions (34c).

The two side portions in the left/right direction of upper coupling portion (37) are bent downward so as to facilitate hand-held operation, and the tip of holding piece (36a) is bent upward for preventing release of the coupling with external holding pieces (13a), (13b). In addition, the upper side portion of connecting piece (38) is bent so that it is inclined relative to the lower side portion for aligning the positions in the vertical direction of the center of the recess of holding piece (36a) and the center of coupling recess (37a).

For extending unit (30) with said constitution, when intracorporeal indwelling equipment A is stretched, first, extender (31) is inserted from coupling hole (16) of external holding member (10) to the lower side of cylindrical member (11), and press-in piece (35b) is inserted into hole (24a) while aligned to coupling member (24) of internal holding member (20). Then, while external holding pieces (13a), (13b) of intracorporeal indwelling equipment A in this state are inserted between holding piece (36a) and hook (36b), extending auxiliary member (32) is assembled with intracorporeal indwelling equipment A and extender (31) while connecting portion (15) is positioned in the recess of holding piece (36a).

Then, the upper surface of holding portion (34b) is pressed by a hand, and a finger engages with the lower surface of upper coupling portion (37) while extending auxiliary member (32) is pulled upward without press-in piece (35b) being removed from hole (24a), and the edge of coupling recess (37a) is coupled to a prescribed step portion (34c), such as step portion (34c) positioned as the second from the lower side. Consequently, as shown in FIG. 5, internal holding member (20) stretches and becomes slender, and cylindrical member (11) and internal holding member (20) become rod-like. In this case, internal holding member (20) is folded so that recesses are formed with creases (27a), (27b) for folding of membrane-like members (23a), (23b) as the boundary, so that the creases (28a), (28b), (28c), (28d) for folding on the side of connecting portion (21) become the crest side (protrusion side).

Also, creases (28a), (28b), (28c), (28d) for folding are folded so that the portions on the side of outer periphery (25a) become the crest side. In this case, as shown in FIG. 2, folding is performed so that the portions indicated by lines (a) become the boundary lines. In this way, internal holding member (20) becomes a fine rod shape, and its plan view becomes small in size as shown in FIG. 11. In this case, not only internal holding member (20), but also cylindrical member (11), is stretched. Also, for external holding pieces (13a), (13b), the bent portion at the tip of holding piece (36a) prevents them from getting out from holding piece (36a). For step portions (34c) of extender (31), intracorporeal indwelling equipment A and extending unit (30) are assembled while protrusions (37b) act to prevent them from getting out of coupling recess (37a).

Then, in this state, intracorporeal indwelling equipment A passes through the fistula (not shown in the figure) formed between the abdominal wall and the stomach wall of the patient. Then, as internal holding member (20) enters the stomach of the patient, while the finger is on the lower surface of upper coupling portion (37), extending auxiliary member (32) is pulled up, and, while coupling recess (37a) is removed from step portion (34c), holding piece (36a) and hooks (36b) are removed from external holding pieces (13a), (13b), and extending auxiliary member (32) is removed from intracorporeal indwelling equipment A. In addition, extender (31) is withdrawn from intracorporeal indwelling equipment A. Then, ribbon-shaped connecting portion (17) is folded and stopper portion (18) is pressed onto the coupling hole (16) of main body (12) to close the coupling hole (16).

As a result, the shape of internal holding member (20) returns to the original state shown in FIG. 1 due to the elasticity, and the upper surface of contact portion (25) comes into contact with the inner surface of the stomach wall. Cylindrical member (11) also returns to the original state. As a result, it is possible to prevent intracorporeal indwelling equipment A from withdrawing from the fistula, and it is kept attached in the abdominal portion of the patient. Also, the portions of the abdominal wall and the stomach wall near the fistula are fixed by intracorporeal indwelling equipment A, and their respective positions do not shift. In this case, the following scheme is preferred: a certain gap is formed between the surface of the abdominal wall and the lower surface of external holding member (10), and a certain freedom is realized between intracorporeal indwelling equipment A and the fistula.

When liquid food, nutrients, or other fluids are to be fed to the patient coupling hole (16) of external holding member (10) is opened, and a tube (not shown in the figure) for feeding the fluid is connected to coupling hole (16). In this state, the fluid enters from the end opening of the fluid feeding tube into the fluid feeding tube. As a result, the fluid substance is fed from the fluid feeding tube via coupling hole (16) and the feeding flow path (11a) into the stomach of the patient. In this case, the fluid flows out from the lower end opening of cylindrical member (11) and from the interior of internal holding member (20) through the portion between ribbon-shaped members (22a), (22b) and into the stomach. After use, the fluid feeding tube is removed from external holding member (10), and coupling hole (16) is closed.

Also, when it is necessary to make an exchange due to changes in the properties, such as stretching, of intracorporeal indwelling equipment A, etc., after use for a prescribed period of time, extender (31) and extending auxiliary member (32) are attached by means of said process to intracorporeal indwelling equipment A while it is kept in the abdomen of the patient. In this case, if stretching of intracorporeal indwelling equipment A occurs, coupling recess (37a) of extending auxiliary member (32) is coupled to step portion (34c) of the third or larger numbered one counted from the lower side. As a result, it is possible to have internal holding member (20) in a fine size appropriate for withdrawals. In this way, while internal holding member (20) is in the slender state, intracorporeal indwelling equipment A can be withdrawn from the body of the patient together with extender (31) and extending auxiliary member (32). Then, new intracorporeal indwelling equipment A is attached to the body of the patient using the aforementioned process.

When said intracorporeal indwelling equipment A is exchanged, a guide wire is fed through lumen (33a) of extender (31), and, after the used intracorporeal indwelling equipment A is removed from the fistula, the guide wire is left within the abdominal and stomach walls. As a result, while the positions of abdominal wall and stomach wall are kept constant, the new intracorporeal indwelling equipment A can be attached in the fistula. Also, in this case, since insertion hole (34d) for extender (31) is tapered with its upper side wider, by positioning the guide wire on the end side of insertion hole (34d), the guide wire does not hamper the operation of extender (31).

In this way, in intracorporeal indwelling equipment A, internal holding member (20) is composed of two flexible ribbon-shaped members (22a), (22b) and membrane-like members (23a), (23b) formed across the upper side between them. Consequently, when intracorporeal indwelling equipment A is mounted on extending unit (30), as ribbon-shaped members (22a), (22b) are stretched to a nearly linear shape, membrane-like members (23a), (23b) collapse to a rod shape as they follow ribbon-shaped members (22a), (22b). On membrane-like members (23a), (23b), notches (26a), (26b) and creases (27a), (27b), (28a), (28b), (28c), (28d) are formed. When internal holding member (20) extends, membrane-like members (23a), (23b) are folded into a prescribed shape by means of notch (26a), etc., and they become smaller as shown in FIG. 11.

For this purpose, it becomes easier to insert internal holding member (20) and cylindrical member (11) into the fistula of the patient or to withdraw them from the fistula of the patient. After intracorporeal indwelling equipment A is inserted into the stomach, the portion of internal holding member (20) on the side of cylindrical member (11) returns to the dome-like shape, and dome-shaped contact portion (25) comes into contact with the stomach wall. Consequently, irritation of the stomach wall by internal holding member (20) or invasion of the stomach wall by the member can be prevented, and it is possible to prevent ulcers and buried bumper syndrome. In addition, because internal holding member (20) is formed in a nearly elliptical shape in the plan view, when the fistula is formed in a nearly elliptical shape, attachment of intracorporeal indwelling equipment A in the fistula becomes easier. Also, because intracorporeal indwelling equipment A can be inserted so that it fits the shape of the fistula, it is possible to reduce resistance during insertion.

### Second Embodiment

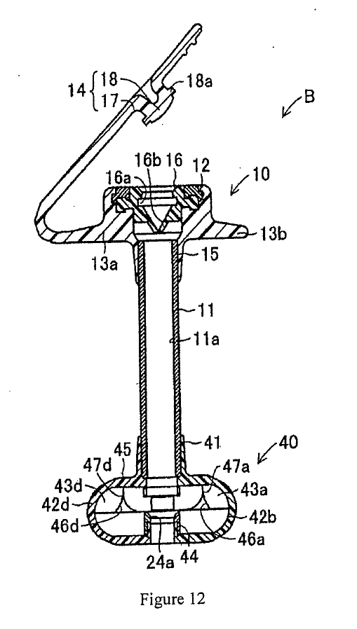

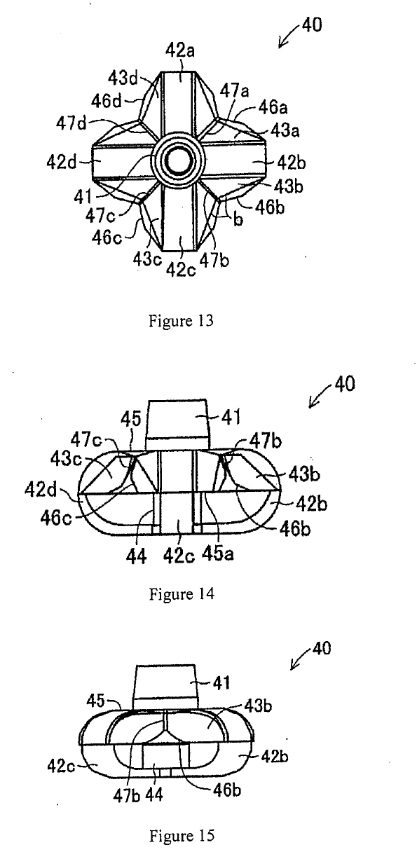

FIG. 12 is a diagram illustrating intracorporeal indwelling equipment B in a second embodiment of the present invention. FIGS. 13-15 illustrate internal holding member (40) of intracorporeal indwelling equipment B. Said internal holding member (40) is composed of cylindrical connecting portion (41), four ribbon-shaped members (42a), (42b), (42c), (42d), four membrane-like members (43a), (43b), (43c), (43d), and coupling member (44). Said connecting portion (41) has the same constitution as that of connecting portion (21) of said intracorporeal indwelling equipment A. Said ribbon-shaped members (42a), (42b), (42c), (42d) are connected to the lower end outer peripheral surface of connecting portion (41). They extend outward from the outer peripheral surface of connecting portion (41) to the four sides and are then bent, and they form a curved shape as they extend from the lower side towards the right, below connecting portion (41).

That is, the lower end portions of ribbon-shaped members (42a), etc., are gathered and fixed on the lower portion of the central axis of connection portion (41). Ribbon-shaped members (42a), (42c) and ribbon-shaped members (42b), (42d) are formed facing each other to depict an ellipse with the minor axis in the longitudinal direction. Also, membrane-like members (43a), (43b), (43c), (43d) are formed in the upper side portions between the various portions adjacent to ribbon-shaped members (42a), (42b), (42c), (42d), and, together with the upper side portions of ribbon-shaped members (42a), etc., they form a nearly dome-shaped contact portion (45).

Notches (46a), (46b), (46c), (46d) are formed at the central portions of outer periphery (45a) of various membrane-like members (43a), etc., respectively. Said notches (46a), etc., are formed in a nearly triangular shape similar to that of notches (26a), etc., of intracorporeal indwelling equipment A. As short, thin, linear portions of the present invention, creases (47a), (47b), (47c), (47d) for folding are formed from the upper end portion of notches (46a), etc., to the lower end portion of connecting portion (41).

In this case, the shape of membrane-like members (43a), (43b), (43c), (43d) is such that creases (47a), (47b), (47c), (47d) become the trough side, and the portions of lines (b) that connect the portions of creases (47a), etc., for folding on the side of notches (46a), (46b), (46c), (46d) and the side portions of ribbon-shaped member (42a), etc., in outer periphery (45a) of membrane-like member (43a), etc., become the crest side. Also, on said internal holding member (40), the creases for folding that correspond to the long, thin, linear portions of the present invention are not formed. The constitution of the portion other than intracorporeal indwelling equipment B is the same as intracorporeal indwelling equipment A. Consequently, the same part numbers are adopted to represent the same parts.

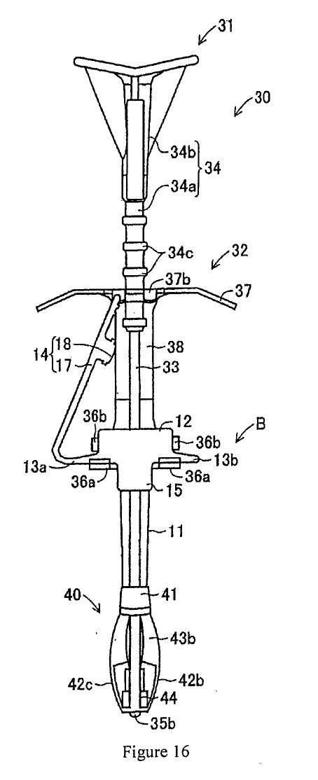

Also, for intracorporeal indwelling equipment B, said extending unit (30) is used and, as shown in FIG. 16, it is put into the fistula of the patient while internal holding member (40) is in the stretched state. In this case, internal holding member (40) is folded such that the lines that connect notches (46a) of membrane-like member (43a), etc., and creases (47a), etc., for folding are on the trough side. Also, in this case, folding is performed for membrane-like member (43a), etc., such that the portions indicated by lines (b) in FIG. 13 (the lines that connect the crossing portions between outer periphery (45a) of membrane-like member (43a), etc., and ribbon-shaped member (42a), etc., and the boundaries between those creases (47a), (47b), (47c), (47d) for folding on the side of outer periphery (45a) and those creases (47a), etc., on the side of connecting portion (41) become the boundary lines on the crest side portion.

For the aforementioned constitution, when internal holding member (40) is inserted into the stomach, said four membrane-like members (43a), etc., expand to the four sides, and they are in contact with the stomach wall in good balance. As a result, it is possible to prevent ulcers and buried bumper syndrome. The other functions and effects of intracorporeal indwelling equipment B are the same as those of input/output interface A.

### Third Embodiment

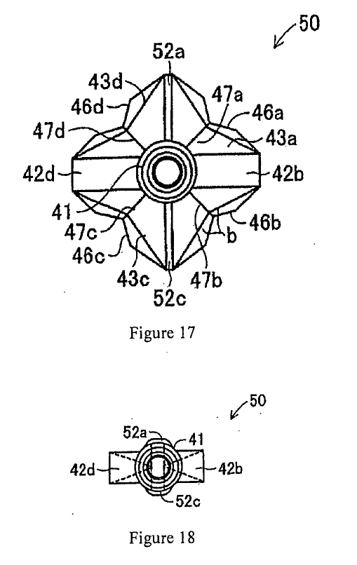

FIG. 17 is a diagram illustrating the upper view of internal holding member (50) having the intracorporeal indwelling equipment in a third embodiment of the present invention. In this embodiment, the internal holding member (50) is composed of narrow (fine) ribbon-shaped members (52a), (52c) as the portions corresponding to ribbon-shaped members (42a), (42c) on internal holding member (40) shown in FIG. 13. The other features of the constitution of the intracorporeal indwelling equipment having internal holding member (50) are the same as those of intracorporeal indwelling equipment B. Consequently, the same part numbers as the aforementioned are adopted, and they will not be explained again. In this case, as internal holding member (50) stretches, the shape in the plan view becomes that shown in FIG. 18. In this case, too, insertion and withdrawal of the intracorporeal indwelling equipment can be easily performed.

The present invention is not limited to the aforementioned embodiments. The present invention may also be changed appropriately. For example, in said embodiments, intracorporeal indwelling equipment A, etc., is made of polyurethane. However, other materials may also be adopted for forming intracorporeal indwelling equipment A, etc., such as polypropylene, silicone, polycarbonate, and other resin materials. Also, in the aforementioned embodiments, intracorporeal indwelling equipment A, etc., have external holding member (10). However, external holding member (10) may be absent from the intracorporeal indwelling equipment, and the cylindrical member may also be made of a long tube.

In addition, for ribbon-shaped member (22a), etc., in intracorporeal indwelling equipment A, etc., appropriate changes may be made in the number, shape, etc., of creases (47a), etc., for folding, such as notch (26a). In said embodiments, the stomach is taken as the organ in the body for attachment of intracorporeal indwelling equipment A. However, the organ is not limited to the stomach. For example, it may also be adopted for the duodenum and the other intestines, renal pelvis, bladder, etc. For example, it may be used to remove urine from the renal pelvis, or for removing other contents from the body.

For the intracorporeal indwelling equipment of the present invention with the aforementioned constitution, the internal holding member is composed of multiple flexible ribbon-shaped members and membrane-like members formed between them. Consequently, when the ribbon-shaped members become similar to a straight line shape (hereinafter to be referred to as a linear shape), the membrane-like members collapse after them into a rod shape. Also, notches are formed on the outer periphery of the membrane-like members, and, when the multiple ribbon-shaped members are stretched into a linear shape, the membrane-like members are folded to a prescribed shape with the notches as the base portions. That is, when the ribbon-shaped members are stretched into linear shape, the membrane-like members are folded with the lines that connect the cylindrical member side portion with the notches as it creases. Also, in this case, the base of the crease refers to the portion from which the crease is formed.

Consequently, when the internal holding member and the cylindrical member are inserted into the fistula of the patient or pulled out from the fistula, for example, when a rod-shaped extender or the like is inserted into the cylindrical member, the bonding portion of the tip of the ribbon-shaped member is pressed by the tip of the extender, so that the internal holding member is folded and becomes a slender rod shape that extends along the extender. Also, in this case, the membrane-like members do not expand in an irregular shape. Instead, together with the ribbon-shaped members, they become a slender rod shape in a prescribed shape. As a result, the intracorporeal indwelling equipment can be inserted into the fistula of the patient or pulled out from the fistula easily.

Also, after the intracorporeal indwelling equipment has been inserted into the prescribed portion, the extender is pulled from the cylindrical member, the portion of the internal holding member on the cylindrical member side recovers the dome shape, and the dome-shaped portion comes into contact with the inner wall surface of the prescribed portion. Consequently, the internal holding member does not irritate the inner wall of the prescribed portion and does not invade the inner wall. As a result, it is possible to prevent generation of ulcers or buried bumper syndrome. Also, the “prescribed portion” in this case refers to any of the organs in the human body, such as stomach, duodenum and other intestines, renal pelvis, bladder, etc.

In this case, the notches may be (inter alia) any of the following types: linear notches extending from the outer periphery of the dome-shaped portion towards the cylindrical member side, notches with a prescribed width on the opening side, notches each with the opening side in contact and closed and with recess formed in a prescribed shape on the inner side, etc. Among these notches, in particular, the notch formed with the width tapered narrower from the outer periphery of the dome-shaped portion towards the cylindrical member side is preferred. As a result, in addition, the membrane-like members can be folded easily. Also, the internal holding member and the cylindrical member can be connected using a cylindrical connecting portion. The connecting portion can be integrally formed to the internal holding member or integrated with the cylindrical member. Also, when the internal holding member and the cylindrical member are integrally formed, there is no need to have the connecting portion.

As another feature of the intracorporeal indwelling equipment of the present invention, each said membrane-like member forms a short, thin, linear portion extending from said notch to the side of said cylindrical member. As a result, by means of the notch and the short, thin, linear portion, the folding direction of the membrane-like members becomes constant. That is, by means of the notch, the starting point of the crease is determined, and the crease extends along the short, thin, linear portion. Consequently, the membrane-like members can deform reliably so that they collapse to the ribbon-shaped member side extending in the linear shape.

As another feature of the intracorporeal indwelling equipment of the present invention, said ribbon-shaped members are four ribbon-shaped members extending from the opening edge of said cylindrical member towards the four sides with prescribed spacing between them around the circumference; on each of the membrane-like members formed between said four ribbon-shaped members, a notch at the center of the outer periphery of said membrane-like member and a short, thin, linear portion extending from said notch towards said cylindrical member are formed. In this case, the following scheme is preferred: the shape of said membrane-like member is such that said short, thin, linear portion is formed on the trough side, and the line connecting the portion of said short, thin, linear portion on said notch side and the portion of the outer periphery of said membrane-like member on said membrane-like member side is on the crest side.

In this way, as the internal holding member is inserted into the prescribed portion, the four ribbon-shaped members extend to the four sides, so that the intracorporeal indwelling equipment can be set in the fistula with good balance. Also, when the intracorporeal indwelling equipment is stretched, the internal holding member always becomes the same slender shape, so that the intracorporeal indwelling equipment can be inserted into the fistula of the patient or pulled out from the fistula of the patient easily.

As yet another feature of the intracorporeal indwelling equipment of the present invention, said membrane-like members, multiple long, thin, linear portions extending from the portions of the outer periphery of said membrane-like members, except for said notches, towards said cylindrical member side are formed with a prescribed spacing between them. As a result, when the ribbon-shaped members are stretched to a linear shape, the membrane-like members are folded along the long, thin, linear portions and they collapse to the ribbon-shaped member side, or they are folded to the inner side of the ribbon-shaped members. As a result, the membrane-like members collapse together with the ribbon-shaped members into a rod shape with a certain regularity.

As yet another feature of the intracorporeal indwelling equipment of the present invention, said ribbon-shaped member are two ribbon-shaped members extending from the portions facing each other on the opening edge of said cylindrical member in the opposite direction, and each membrane-like members formed between said two ribbon-shaped members has a notch formed at the center of the outer periphery of said membrane-like member, a short, thin, linear portion extending from said notch towards said cylindrical member side, and a long, thin, linear portion extending from the portion between said ribbon-shaped members towards said cylindrical member side.

In this case, the following scheme is preferred: the shape of said membrane-like member is such that said short, thin, linear portion becomes the trough portion, and the portion of said long, thin, linear portion on the cylindrical member side forms the crest side, the portion of said membrane-like member on the outer periphery side becomes the crest side, and, at the same time, the lines connecting the boundary portion between the crest side portion and the trough side portion of said long, thin, linear portion and the portions of the outer periphery of said membrane-like member on said notch side and on said ribbon-shaped member side are formed on the crest side.

As a result, when the stomach fistula is formed in a non-circular shape, such as an elliptic shape, attachment of the intracorporeal indwelling equipment in the stomach fistula can be performed easily. That is, by adjusting the orientation of the intracorporeal indwelling equipment to fit the shape of the fistula, it is possible to reduce the resistance when the intracorporeal indwelling equipment is inserted in the fistula. Also, in this case, the shape of the membrane-like members is the same as that when the multiple ribbon-shaped members are not stretched. During the process when the ribbon-shaped members are stretched, the crest side portion of the membrane-like member and the trough side portion of the membrane-like member become different from each other. When the ribbon-shaped members have been stretched, the crest and trough sides become the same as those before stretching of the ribbon-shaped members. As a result, when the intracorporeal indwelling equipment is stretched, the internal holding member always keeps the same slender shape. Consequently, insertion of the intracorporeal indwelling equipment into the fistula of the patient and withdrawing of the intracorporeal indwelling equipment from the fistula of the patient become easier.

Other variations and modifications will be recognized by those of ordinary skill in the art as being within the scope of the present invention.

1. Apparatus for intracorporeal indwelling, in which the intracorporeal indwelling equipment has a cylindrical member to be set in the fistula formed between the skin side wall portion on the body of the patient and the wall portion of the prescribed portion in the body of the patient, and an internal holding member that is connected to the tip of said cylindrical member and is set on the inner side of said prescribed portion, and it is for the fluid flow between the exterior of said patient and the interior of said prescribed portion;
said internal holding member being composed of multiple flexible ribbon-shaped members, each bending from the opening edge at the tip of said cylindrical member towards the outer side and bonded to each other at their tips, and flexible membrane-like members set on the portions of said multiple ribbon-shaped members on said cylindrical member side and forming a dome-like portion together with said multiple ribbon-shaped members; and wherein a notch is formed on the outer periphery of the membrane-like members of said dome-like portion; when said multiple ribbon-shaped members are stretched so that the tip bonding portions of said ribbon-shaped members separate from said cylindrical member, said membrane-like members are folded, with said notch taken as the base of the crease.

2. Apparatus claimed in claim 1 wherein each of said membrane-like members forms a short, thin, linear portion extending from said notch to the side of said cylindrical member.

3. Apparatus claimed in claim 1 wherein said ribbon-shaped members are four ribbon-shaped members extending from the opening edge of said cylindrical member towards the four sides with prescribed spacing between them around the circumference; on each of the membrane-like members formed between said four ribbon-shaped members, a said notch at the center of the outer periphery of each said membrane-like member and a short, thin, linear portion extending from said notch towards said cylindrical member are formed.

4. Apparatus claimed in claim 3 wherein the shape of each said membrane-like member is such that said short, thin, linear portion is formed on the trough side, and the line connecting the portion of said short, thin, linear portion on said notch side and the portion of the outer periphery of each said membrane-like member on said membrane-like member side is on the crest side.

5. Apparatus claimed in claim 1 wherein on said membrane-like members, multiple long, thin, linear portions extending from the portions of the outer periphery of said membrane-like members, except for said notches towards said cylindrical member side, are formed with a prescribed spacing between them.

6. Apparatus claimed in claim 5 wherein said ribbon-shaped members are two ribbon-shaped members extending from the portions facing each other on the opening edge of said cylindrical member in the opposite direction, and each membrane-like member formed between said two ribbon-shaped members has a said notch formed at the center of the outer periphery of each said membrane-like member, a short, thin, linear portion extending from said notch towards said cylindrical member side, and a long, thin, linear portion extending from the portion between said ribbon-shaped members towards said cylindrical member side.

7. Apparatus claimed in claim 6 wherein the shape of each said membrane-like member is such that said short, thin, linear portion becomes the trough portion, and the portion of said long, thin, linear portion on the cylindrical member side forms the crest side, the portion of each said membrane-like member on the outer periphery side becomes the crest side, and, at the same time, the lines connecting the boundary portion between the crest side portion and the trough side portion of said long, thin, linear portion and the portions of the outer periphery of each said membrane-like member on said notch side and on said ribbon-shaped member side are formed on the crest side.

8. Intracorporeal indwelling equipment comprising a tube member for insertion into a patient and an interior fixing member attached to a distal end of the tube member at a connection region, the interior fixing member comprising a plurality of resilient support members connecting a coupling member to said connection region characterized in that said interior fixing member further comprises a plurality of foldable membrane-like members attached to and between said support members.

9. The intracorporeal indwelling equipment according to claim 8 wherein said membrane-like members are arranged between upper portions of said support members.

10. The intracorporeal indwelling equipment according to claim 8 wherein said support members together with said membrane-like members form a dome arrangement for contacting with an organ wall of a patient's organ.

11. The intracorporeal indwelling equipment according to claim 8 wherein said membrane-like members include pre-formed creases in pre-determined positions for controlling an unfolding and folding action of said membrane-like members when a force is applied or removed from said coupling member.

12. The intracorporeal indwelling equipment according to claim 8 wherein said interior fixing member comprises two support members and two membrane-like members.

13. The intracorporeal indwelling equipment according to claim 8 wherein said interior fixing member comprises four support members and four membrane-like members.

14. The intracorporeal indwelling equipment according to claim 13 wherein two of said support members are relatively thin in relation to the other two support members.

15. The intracorporeal indwelling equipment according to claim 8 wherein said membrane-like members include a notch region in a medial region of a free edge of said membrane-like member.

16. The intracorporeal indwelling equipment according to claim 15 wherein each membrane-like member further includes at least one ridge region extending from said connection region to a peripheral edge of said membrane-like member.

17. The intracorporeal indwelling equipment according to claim 15 wherein a crease region extends from an apex of said notch region to said connection region.

18. The intracorporeal indwelling equipment according to claim 8 wherein said equipment is a gastrostomy tube for insertion into a patient's stomach.

19. A method of inserting an intracorporeal indwelling equipment into a patient comprising the steps of:
(i) providing an equipment according to claim 1;
(ii) placing an internal holding member of the equipment in an insertion state in which membrane-like members of the holding member are in a folded state and ribbon-like members of the holding member are in a stretched state;
(iii) introducing the equipment into the patient; and
(iv) placing the internal holding member in a deployed state in which the membrane like members are unfolded and the ribbon-like members are in a substantially relaxed state.

20. A method of inserting an intracorporeal indwelling equipment into a patient comprising the steps of:
(i) providing an equipment according to claim 8;
(ii) placing an internal fixing member of the equipment in an insertion state in which membrane-like members of the holding member are in a folded state and support members of the fixing member are in a stretched state;
(iii) introducing the equipment into the patient; and
(iv) placing the internal fixing member in a deployed state in which the membrane like members are unfolded and the support members are in a substantially relaxed state.

21. An intracorporeal indwelling equipment comprising an extension tube and an internal holding member attached to a peripheral end of said extension tube by a connection region, wherein said internal holding member comprises a coupling member and a plurality of support members connecting said coupling member to said connection region and a plurality of skirt members extending between said support members and wherein each said skirt member includes a notch region at a peripheral edge thereof and wherein a fold region extends from an apex of said notch region towards said connection region.

22. The intracorporeal indwelling equipment according to claim 21 wherein each said skirt member includes at least one ridge region extending from the peripheral edge of said skirt member towards the connection region, the ridge region being arranged between the fold region and a respective support member.

23. The intracorporeal indwelling equipment according to claim 21 wherein said support members are elastically resilient.

24. The intracorporeal indwelling equipment according to claim 21 wherein said skirt members each comprise an elastic membrane.

25. The intracorporeal indwelling equipment according to claim 21 wherein said internal holding member comprises two support members.

26.-28. (canceled)
